(19) United States
(12) Patent Application Publication
(10) Pub. No.: US 2012/0157380 A1
(43) Pub. Date: Jun. 21, 2012

(54) PEGYLATED HUMAN APOA-1 AND PROCESS FOR PRODUCTION THEREOF

(76) Inventors: Nan Wang, Flushing, NY (US); Alan R. Tall, Cresskill, NJ (US)

(21) Appl. No.: 13/376,350

(22) PCT Filed: Jun. 3, 2010
(86) PCT No.: PCT/US2010/001631

§ 371 Date: Mar. 1, 2012

Related U.S. Application Data
(60) Provisional application No. 61/217,922, filed on Jun. 5, 2009.
(60) Provisional application No. 61/278,770, filed on Oct. 8, 2009.

(51) Int. Cl. A61K 38/17; A61P 3/06; A61P 29/00; C07K 14/775; C07K 1/107

(52) U.S. Cl. 514/7.4; 530/359; 514/21.2

(57) ABSTRACT

An isolated mono-pegylated apolipoproteinA-1, compositions comprising such, and methods of treating cardiovascular diseases using mono-pegylated apolipoproteinA-1 are provided as well as processes for making isolated mono-pegylated apolipoproteinA-1 and compositions containing such.

This application claims priority of U.S. Provisional Applications Nos. 61/278,770, filed Oct. 8, 2009 and 61/217,922, filed Jun. 5, 2009, the entire content of each of which is hereby incorporated by reference herein.

The work disclosed herein was made with government support under grant no. H1 54591 from the National Institutes of Health. Accordingly, the U.S. Government has certain rights in this invention.

Throughout this application various publications and published patents are referenced. The disclosures of these publications in their entireties are hereby incorporated by reference into this application in order to more fully describe the state of the art to which this invention pertains.

### BACKGROUND

Plasma HDL cholesterol levels are inversely correlated to the risk of cardiovascular disease, suggesting a protective role. In humans, recombinant HDL has been tested in clinical trials for its potential anti-atherosclerotic properties and these studies have indeed shown a protective role of HDL against development of atherosclerosis.

Currently, purified human apolipoprotein A-1 (apoA-I), either in the recombinant or native form, is used in combination with phospholipids and bile salts in order to generate recombinant human HDL particles. While the amphipathic nature of bile salt facilitates recombinant HDL particle formation, the residual bile salt in the recombinant HDL preparation can cause adverse effects in humans and therefore limit the quantity of usable recombinant HDL particles. Since apoA-I and HDL are the major human plasma apolipoprotein and lipoprotein species, it is critical to be able to achieve sufficient levels of plasma recombinant HDL in humans. The initial levels of plasma recombinant HDL and maintenance of its sufficient therapy concentration over appropriate period of time could determine the efficacy of the HDL therapy.

The mechanism responsible for turnover of plasma HDL is still poorly defined. Liver and kidney are the major organs involved in regulation of HDL catabolism. If the clearance of plasma HDL or its apolipoproteins can be reduced, it is likely that sufficient therapeutic concentration could be achieved with a reduced dose of HDL.

### SUMMARY OF THE INVENTION

An isolated mono-pegylated apolipoproteinA-1.

A composition comprising a carrier and a total molar amount of mono-pegylated apolipoproteinA-1, wherein the total molar amount of mono-pegylated apolipoproteinA-1 is more than 50% relative to the total molar amount of apolipoproteinA-1, wherein the total molar amount of apolipoproteinA-1 includes mono-pegylated apolipoproteinA-1, multi-pegylated apolipoproteinA-1, and non-pegylated apolipoproteinA-1.

A composition comprising a high-density lipoprotein particle which comprises a pegylated apolipoproteinA-1, wherein the composition is essentially free of bile salts.

An isolated mono-pegylated apolipoproteinA-1 analog, comprising a single apolipoproteinA-1 analog molecule covalently bonded to a single polyethylene glycol molecule.

A composition comprising mono-pegylated apolipoproteinA-1 analog, wherein the mono-pegylated apolipoproteinA-1 analog comprises a single apolipoproteinA-1 analog molecule covalently bonded to a single polyethylene glycol molecule, and wherein the composition is substantially free of multi-pegylated apolipoproteinA-1.

A high-density lipoprotein particle essentially free of bile salts comprising a pegylated apolipoproteinA-1 analog.

A process for preparing an isolated mono-pegylated apolioprotein-A1:

(a) admixing apolipoproteinA-1 with a source of polyethylene gylcol under conditions permitting formation of a covalent bond between the apolipoproteinA-1 and the polyethylene glycol; and
(b) separating mono-pegylated apolioprotein-A1 from the product of step (a) or quenching step (a) before production of multi-pegylated apolipoproteinA-1 occurs, so as to thereby prepare mono-pegylated apolioprotein-A1.

A process for making a composition comprising a carrier and a total molar amount of mono-pegylated apolipoproteinA-1, wherein the total molar amount of mono-pegylated apolipoproteinA-1 is more than 50% relative to the total molar amount of apolipoproteinA-1, wherein the total molar amount of apolipoproteinA-1 includes mono-pegylated apolipoproteinA-1, multi-pegylated apolipoproteinA-1, and non-pegylated apolipoproteinA-1, the process comprising: (a) admixing apolipoproteinA-1 with a source of polyethylene gylcol under conditions permitting formation of a covalent bond between the apolipoproteinA-1 and the polyethylene glycol; and (b) quenching step (a) so as to control the amount of multi-pegylated apolipoproteinA-1 produced, so as to thereby prepare the composition

A process for increasing the plasma activity of an apolipoproteinA-1 comprising: (a) obtaining a apolipoproteinA-1; and (b) covalently bonding a single polyethylene gylcol molecule thereto, so as to thereby make a monopegylated apolipoproteinA-1 having increased plasma activity relative to the apolipoproteinA-1

A method of treating an inflammatory vascular disease in a subject comprising administering to the subject an amount of the compounds or the compositions described herein effective to treat the inflammatory vascular disease in the subject.

A method of treating an inflammatory vascular disease in a subject comprising administering to the subject an amount of the high-density lipoprotein particles described herein effective to treat the inflammatory vascular disease in the subject

A method of treating a dyslipidemia in a subject comprising administering to the subject an amount of the high-density lipoprotein particles, the compounds, or the compositions described herein, effective to treat the dyslipidemia in the subject.

A method of increasing plasma high-density lipoprotein levels in a subject comprising administering to the subject an amount of the high-density lipoprotein particles, the compounds, or the compositions described herein, effective to increase plasma high-density lipoprotein levels in the subject.

A method of promoting cholesterol efflux from macrophage foam cells in a subject comprising administering to the subject an amount of the high-density lipoprotein particles, the compounds, or the compositions described herein, effective to promote cholesterol efflux from macrophage foam cells in the subject.

### BRIEF DESCRIPTION OF THE FIGURES

FIG. 1

Pegylation of human apoA-I. Purified human apoA-I was pegylated as described hereinbelow. After pegylation, the apoA-I preparation incubated without (lane A1) or with (lane A2) activated PEG for −16 hours at 4° C. was analyzed by SDS-PAGE as shown. Incubation at room temperature for 16 hours results in multipegylation of apoA-I, as indicated by multiple species of PEG-apoA-I in 1B (B1, 2 without PEG and B3, 4 with PEG).

FIG. 2

Cholesterol efflux from macrophage foam cells. Mouse peritoneal macrophage were generated and labeled by incubation of the macrophage cells with 50 g/ml acetyl-LDL and 1 μCi/ml [³H] cholesterol incorporated into the acetyl-LDL preparation overnight. Cholesterol efflux was initiated by adding apoA-I or PEG-apoA-I at the indicated concentration after washing the cells. After 3 hours, the media and cells were collected and the media and cellular radioactivity were determined. Percentage efflux was determined as media count/(media+cellular count)×100.

FIG. 3

ApoA-I clearance in mice. Iodinated [¹²⁵I] apoA-I (mixture of apoA-I and PEG-apoA-1) was injected into the blood circulation via the tail vein of the mouse. The blood samples were taken periodically over 24 hours and subjected to SDS-PAGE. Autoradiogram of results shown.

FIG. 4

Distribution of PEG-apoA-I in plasma. PEG-apoA-I was injected into the mouse via the tail vein. 30 minutes after injection, the plasma samples were taken from the mouse and subjected to density gradient centrifugation (d-1.21). The fractions of the centrifugation were then analyzed by SDS-PAGE and immunoblotting.

### DETAILED DESCRIPTION OF THE INVENTION

An isolated mono-pegylated apolipoproteinA-1.

In an embodiment the isolated mono-pegylated apolipoproteinA-1 comprises consecutive amino acids having the sequence set forth in SEQ ID NO:1. In an embodiment the apolipoproteinA-1 comprises consecutive amino acids encoded by the nucleic acid sequence set forth in SEQ ID NO:2 or SEQ ID NO:3.

In an embodiment the polyethylene glycol has a molecular weight of between 19,000 and 21,000. In an embodiment the polyethylene glycol has a molecular weight of 20,000.

In an embodiment in the apolipoproteinA-1 is N-terminally mono-pegylated.

In an embodiment the apolipoproteinA-1 is A-1 Milano (Arg173Cys)or the variant (Arg173Pro). In an embodiment the apolipoproteinA-1 is apolipoproteinA-1 Paris.

A composition comprising a carrier and a total molar amount of mono-pegylated apolipoproteinA-1, wherein the total molar amount of mono-pegylated apolipoproteinA-1 is more than 50% relative to the total molar amount of apolipoproteinA-1, wherein the total molar amount of apolipoproteinA-1 includes mono-pegylated apolipoproteinA-1, multi-pegylated apolipoproteinA-1, and non-pegylated apolipoproteinA-1.

In an embodiment of the composition the apolipoproteinA-1 comprises consecutive amino acids having the sequence set forth in SEQ ID NO:1. In an embodiment of the composition the apolipoproteinA-1 comprises consecutive amino acids encoded by the nucleic acid sequence set forth in SEQ ID NO:2 or SEQ ID NO:3. In an embodiment of the composition the polyethylene glycol has a molecular weight of between 19,000 and 21,000. In an embodiment of the composition the polyethylene glycol has a molecular weight of 20,000. In an embodiment of the composition the apolipoproteinA-1 is N-terminally mono-pegylated. In an embodiment of the composition the apolipoproteinA-1 is A-1 Milano (Arg173Cys)or the variant (Arg173Pro).

In an embodiment of the composition at least 85% of the total amount of mono-pegylated apolipoproteinA-1 and multi-pegylated apolipoproteinA-1 in the composition is mono-pegylated apolipoproteinA-1. In an embodiment of the composition at least 90% of the total amount of mono-pegylated apolipoproteinA-1 and multi-pegylated apolipoproteinA-1 in the composition is mono-pegylated apolipoproteinA-1. In an embodiment of the composition at least 95% of the total amount of mono-pegylated apolipoproteinA-1 and multi-pegylated apolipoproteinA-1 in the composition is mono-pegylated apolipoproteinA-1. In an embodiment of the composition at least 99% of the total amount of mono-pegylated apolipoproteinA-1 and multi-pegylated apolipoproteinA-1 in the composition is mono-pegylated apolipoproteinA-1.

In an embodiment of the composition the apolipoproteinA-1 comprises consecutive amino acids having the sequence set forth in SEQ ID NO:1. In an embodiment of the composition the apolipoproteinA-1 comprises consecutive amino acids encoded by the nucleic acid sequence set forth in SEQ ID NO:2 or SEQ ID NO:3. In an embodiment of the composition the apolipoproteinA-1 is A-1 Milano (Arg173Cys)or the variant (Arg173Pro).

In an embodiment of the composition the polyethylene glycol of the mono-pegylated and/or multi-pegylated apolipoproteinA1 has a molecular weight of between 19,000 and 21,000. In an embodiment of the composition the polyethylene glycol has a molecular weight of 20,000. In an embodiment of the composition the mono-pegylated apolipoproteinA-1 is N-terminally mono-pegylated.

In an embodiment the composition comprises a pharmaceutically acceptable carrier. In an embodiment the pharmaceutically acceptable carrier comprises sucrose-mannitol and a phosphate buffer. In an embodiment the composition or carrier has a pH of 7.0 to 7.8. In an embodiment the pH is about 7.5.

A composition comprising a high-density lipoprotein particle which comprises a pegylated apolipoproteinA-1, wherein the composition is essentially free of bile salts.

In an embodiment of the particle the pegylated apolipoproteinA-1 is mono-pegylated apolipoproteinA-1 comprising a single apolipoproteinA-1 molecule covalently bonded to a single polyethylene glycol molecule. In an embodiment of the particle the pegylated apolipoproteinA-1 comprises consecutive amino acids having the sequence set forth in SEQ ID NO:1. In an embodiment of the particle the pegylated apolipoproteinA-1 comprises consecutive amino acids encoded by the nucleic acid sequence set forth in SEQ ID NO:2 or SEQ ID NO:3. In an embodiment of the particle the apolipoproteinA-1 of the pegylated apolipoproteinA-1 is A-1 Milano (Arg173Cys) or the variant (Arg173Pro).

In an embodiment of the particle the polyethylene glycol of the pegylated apolipoproteinA-1 has a molecular weight of between 19,000 and 21,000. In an embodiment of the particle the pegylated apolipoproteinA-1 has a molecular weight of 20,000. In an embodiment of the particle the pegylated apolipoproteinA-1 is N-terminally mono-pegylated.

In an embodiment of the particle the high-density lipoprotein particle is present in a composition comprising a pharmaceutically acceptable carrier. In an embodiment the pharmaceutically acceptable carrier comprises sucrose-mannitol and a phosphate buffer. In an embodiment the pH of the composition or carrier is 7.0 to 7.8. In an embodiment the pH is about 7.5.

An isolated mono-pegylated apolipoproteinA-1 analog, comprising a single apolipoproteinA-1 analog molecule covalently bonded to a single polyethylene glycol molecule.

A composition comprising mono-pegylated apolipoproteinA-1 analog, wherein the mono-pegylated apolipoproteinA-1 analog comprises a single apolipoproteinA-1 analog molecule covalently bonded to a single polyethylene glycol molecule, and wherein the composition is substantially free of multi-pegylated apolipoproteinA-1.

A high-density lipoprotein particle essentially free of bile salts comprising a pegylated apolipoproteinA-1 analog.

A process for preparing a mono-pegylated apolioprotein-A1, comprising a single apolipoproteinA-1 molecule covalently bonded to a single polyethylene glycol molecule, the method comprising:
admixing apolipoproteinA-1 with a source of polyethylene gylcol under conditions permitting formation of a covalent bond between the apolipoproteinA-1 and the polyethylene glycol; and separating mono-pegylated apolioprotein-A1 from the product of step (a) or quenching step (a) before production of multi-pegylated apolipoproteinA-1 occurs,
so as to thereby prepare mono-pegylated apolioprotein-A1.

In an embodiment the source of polyethylene gylcol is methoxypoly(ethylene glycol). In an embodiment the source of polyethylene gylcol and the apolipoproteinA-1 are admixed in a 8:1 to 12:1 ratio. In an embodiment the source of polyethylene gylcol and the apolipoproteinA-1 are admixed in a 9:1 to 10:1 ratio. In an embodiment erein the source of polyethylene gylcol and the apolipoproteinA-1 are admixed in a 10:1 ratio. In an embodiment the conditions comprise admixing for 8-14 hours at 4° C.

In an embodiment in step (b) the quenching comprises adding 5 mM-75 mM glycine to the products of step (a) 8-14 hours after step (a) is initiated. In an embodiment 48 mM-52 mM glycine is added. In an embodiment 50 mM of glycine is added. In an embodiment 8 mM-12 mM glycine is added. In an embodiment 10 mM of glycine is added.

In an embodiment the mono-pegylated apolipoproteinA-1 is separated using a size separation technique. In an embodiment in step (b) the admixture is quenched and further comprising a step of separation of mono-pegylated apolipoproteinA-1. In an embodiment the mono-pegylated apolipoproteinA-1 is separated using a size separation technique.

A process for making a composition comprising mono-pegylated apolipoproteinA-1 and multi-pegylated apolipoproteinA-1, wherein, of the total amount of mono-pegylated apolipoproteinA-1 and multi-pegylated apolipoproteinA-1 in the composition, at least 75% is mono-pegylated apolipoproteinA-1 the method comprising: (a) admixing apolipoproteinA-1 with a source of polyethylene gylcol under conditions permitting formation of a covalent bond between the apolipoproteinA-1 and the polyethylene glycol (b) quenching step (a) so as to control the amount of multi-pegylated apolipoproteinA-1 produced, so as to thereby prepare the composition.

In an embodiment the source of polyethylene gylcol is methoxypoly(ethylene glycol). In an embodiment of the total amount of mono-pegylated apolipoproteinA-1 and multi-pegylated apolipoproteinA-1 in the composition, at least 75% is mono-pegylated apolipoproteinA-1. In an embodiment at least 85% of the total amount of mono-pegylated apolipoproteinA-1 and multi-pegylated apolipoproteinA-1 in the composition is mono-pegylated apolipoproteinA-1. In an embodiment at least 90% of the total amount of mono-pegylated apolipoproteinA-1 and multi-pegylated apolipoproteinA-1 in the composition is mono-pegylated apolipoproteinA-1. In an embodiment at least 95% of the total amount of mono-pegylated apolipoproteinA-1 and multi-pegylated apolipoproteinA-1 in the composition is mono-pegylated apolipoproteinA-1. In an embodiment at least 99% of the total amount of mono-pegylated apolipoproteinA-1 and multi-pegylated apolipoproteinA-1 in the composition is mono-pegylated apolipoproteinA-1.

In an embodiment the source of polyethylene gylcol and the apolipoproteinA-1 are admixed in a 8:1 to 12:1 ratio. In an embodiment the source of polyethylene gylcol and the apolipoproteinA-1 are admixed in a 9:1 to 10:1 ratio. In an embodiment the source of polyethylene gylcol and the apolipoproteinA-1 are admixed in a 10:1 ratio.

In an embodiment herein the conditions comprise admixing for 8-14 hours at 4° C.

In an embodiment in step (b) the quenching comprises adding 5 mM-75 mM glycine to the products of step (a) 8-14 hours after step 9a) is initiated. In an embodiment 48 mM-52 mM glycine is added. In an embodiment 8 mM-12 mM glycine is added. In an embodiment 50 mM glycine is added. In an embodiment 10 mM glycine is added.

In an embodiment the polyethylene glycol has a molecular weight of between 19,000 and 21,000. In an embodiment the polyethylene glycol has a molecular weight of 20,000.

A process for increasing the plasma activity of an apolipoproteinA-1 comprising: (a) obtaining a apolipoproteinA-1; and (b) covalently bonding a single polyethylene gylcol molecule thereto, so as to thereby make a monopegylated apolipoproteinA-1 having increased plasma activity relative to the apolipoproteinA-1.

In an embodiment the polyethylene gylcol is derived from methoxypoly(ethylene glycol). In an embodiment the polyethylene gylcol and the apolipoproteinA-1 are admixed in a 8:1 to 12:1 ratio. In an embodiment the polyethylene gylcol and the apolipoproteinA-1 are admixed in a 9:1 to 10:1 ratio. In an embodiment the polyethylene gylcol and the apolipoproteinA-1 are admixed in a 10:1 ratio.

In an embodiment the apolipoproteinA-1 has the single polyethylene gylcol molecule covalently bonded thereto by admixing apolipoproteinA-1 and polyethylene gylcol for 8-14 hours at 4° C. and quenching thereafter with adding 25 mM-75 mM glycine. In an embodiment the concentration of 48 mM-52 mM glycine is used.

In an embodiment the increase in plasma activity is effected by the increase in the circulatory half-life of the apolipoproteinA-1 resulting from mono-pegylation of the apolipoproteinA-1.

A method of treating an inflammatory vascular disease in a subject comprising administering to the subject an amount of the compounds or the compositions described herein effective to treat the inflammatory vascular disease in the subject.

In an embodiment the inflammatory vascular disease is an atheroma or atherosclerosis. In an embodiment the inflammatory vascular disease is atherothrombotic cardiovascular disease.

A method of treating an inflammatory vascular disease in a subject comprising administering to the subject an amount of the high-density lipoprotein particles described herein effective to treat the inflammatory vascular disease in the subject.

In an embodiment the inflammatory vascular disease is an atheroma or atherosclerosis. In an embodiment the inflammatory vascular disease is atherothrombotic cardiovascular disease.

A method of treating a dyslipidemia in a subject comprising administering to the subject an amount of the high-density lipoprotein particles, the compounds, or the compositions described herein, effective to treat the dyslipidemia in the subject.

A method of increasing plasma high-density lipoprotein levels in a subject comprising administering to the subject an amount of the high-density lipoprotein particles, the compounds, or the compositions described herein, effective to increase plasma high-density lipoprotein levels in the subject.

A method of promoting cholesterol efflux from macrophage foam cells in a subject comprising administering to the subject an amount of the high-density lipoprotein particles, the compounds, or the compositions described herein, effective to promote cholesterol efflux from macrophage foam cells in the subject.

“Administering” an agent can be effected or performed using any of the various methods and delivery systems known to those skilled in the art. The administering can be, for example, intravenous, oral, intramuscular, intravascular, intra-arterial, intracoronary, intramyocardial and subcutaneous.

The term “HDL particle” encompasses HDL particles known to those skilled in the art, including pre-β HDL, β HDL, HDL₂ (including HDL₂ₐ and HDL_{2b}), and HDL₃.

An “isolated mono-pegylated apolipoproteinA-1” is an apolipoproteinA-1 having a single PEG molecule bound thereto via a covalent bond and which is separate from multi-pegylated apolipoproteinA-1 and/or non-pegylated apolipoproteinA-1.

“Mono-pegylated”, as applied to a PEG-derivatized molecule, shall mean the attachment of one, but no more than one, polyethylene glycol molecule via a covalent bond to the molecule being derivatized.

“Multi-pegylated” as applied to a PEG-derivatized molecule, shall mean the attachment of more than one, for example two, three, four or more, polyethylene glycol molecules, each via a covalent bond, to the molecule being derivatized. For example, an apolipoproteinA-1 having two polyethylene glycol molecules attached thereto is multi-pegylated.

“N-terminally pegylated”, as applied to a PEG-derivatized peptide, polypeptide, or protein, shall mean the attachment of a polyethylene glycol molecule via a covalent bond to the N-terminal region of the peptide, polypeptide, or protein being derivatized, the N-terminal region being the N-terminal 50%, 25% or 10% of the peptide, polypeptide, or protein. In an embodiment, N-terminally pegylated shall mean the attachment of a polyethylene glycol molecule via a covalent bond to the N-terminal residue (i.e. the N-terminus).

“Inflammatory vascular disease” shall mean a disease of the vascular or cardiovascular system of a human comprising an inflammatory response in a tissue of the vascular or cardiovascular system, for example a blood vessel thereof. In an embodiment, the disease is diabetic cardiovascular disease.

“Dyslipidemia” is a pathological state marked by the elevation of plasma cholesterol in a subject, triglycerides (TGs), or both, or a low high density lipoprotein level that contributes to the development of atherosclerosis. Causes may be primary (genetic) or secondary. Levels of or serum cholesterol>240 mg/dL (>6.2 mmol/L) are indicative of a dyslipidemia.

“Essentially free of bile salts” shall mean, with regard to a composition, a composition wherein the levels of bile salts, including bile salts based on the bile acids cholic, deoxycholic, chenodeoxycholic, and lithocholic acids, are not present or are undetectable by conventional methods. Compositions that are prepared without the use of bile salts but which may nonetheless contain trace amounts of bile salts fall into this category.

“Essentially free of multi-pegylated apolipoproteinA-1” shall mean, with regard to a composition, a composition wherein multi-pegylated apolipoproteinA-1 is not present or is at a level undetectable by conventional methods. The apolipoproteinA-1 (apoA-I), which is pegylated as described hereinbelow, may be wild-type apolipoprotein or a variant thereof which is A-1 Milano (Arg173Cys)or the variant (Arg173Pro), and those variants described in U.S. Pat. No. 7,223,726, Oda et al., issued May 29, 2007 which is herein incorporated by reference in its entirety. The wild type human ApoA-I protein sequence is found under GenBank Accession No. NP_{—}000030 (SEQ ID NO:1). The apolipoproteinA-1 may be that encoded by NCBI Reference Sequence: NM_{—}000039.1 (SEQ ID NO:2) or as encoded by GenBank: BC005380.1 (SEQ ID NO:3) (apolipoprotein cDNA).

In the compositions or methods of the invention regarding a “mono-pegylated apolipoproteinA-1 analog”, the analog has from 70% to 99% sequence identity to the sequence set forth in SEQ ID NO:1, or encoded by SEQ ID NOs:2 or 3. In addition, the mono-pegylated apolipoproteinA-1 analog is able to mediate cholesterol efflux, from e.g. human macrophage foam cells, as measured by any of the routine assays known to those in the art, including the assay described hereinbelow.

ApolipoproteinA-1 purification methods are described in U.S. Pat. Nos. 6,090,921 and 6,423,830 which use an anion-exchange chromatography gel to purify ApoA, and are hereby incorporated by reference in their entirety.

In an embodiment, the pegylated apolipoproteinA-1 is administered at 1 mg/kg to about 100 mg/kg of a subject's body weight. In an embodiment, the pegylated apolipoproteinA-1 is administered at 5 mg/kg to 80 mg/kg of body weight. In an embodiment, the pegylated apolipoproteinA-1 is administered at 10 mg/kg to 60 mg/kg of body weight, wherein the milligram amount refers to the total amount of apolipoproteinA-1 content. For example, an amount of 10 mg/kg means a total amount of pegylated apoliprotein A-1 wherein the total amount includes 10 mg of apoliproteinA-1. In an embodiment, the pegylated apolipoproteinA-1 is administered at 11 mg/kg to 45 mg/kg of body weight. In an embodiment, the pegylated apolipoproteinA-1 is administered at 12 mg/kg to 18 mg/kg of body weight. A subject can be administered a single type or any combination of the mono-pegylated apoA-1 molecules described herein, wherein the total amount of the single type of mono-pegylated apoA-1 or the summed amount of the different types of mono-pegylated apoA-1 amounts to the doses set forth herein. Doses can be administered of differing amounts during treatment, for example one or two high bolus doses at the beginning of treatment followed by lower maintenance doses.

In one embodiment, the therapeutically or prophylactically effective amount is from about 1 mg of agent/subject to about 1 g of agent/subject per dosing.

In another embodiment, the therapeutically or prophylactically effective amount is from about 10 mg of agent/subject to 500 mg of agent/subject. In a further embodiment, the therapeutically or prophylactically effective amount is from about 50 mg of agent/subject to 200 mg of agent/subject. In a further embodiment, the therapeutically or prophylactically effective amount is about 100 mg of agent/subject. In still a further embodiment, the therapeutically or prophylactically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject.

In an embodiment, the pharmaceutical formulation or the pegylated apolipoproteinA-1 is administered to a subject once weekly for about 6 months, about 5 months, about 4 months, about 3 months, about 2 months or about 1 month. In an embodiment, the pharmaceutical formulation or the pegylated apolipoproteinA-1 is administered about every day, about every other day, about every 3 days, about every 4 days, about every 5 days, about every 6 days, about every 7 days, about every 8-10 days or about every 11-14 days.

In an embodiment, the pegylated apolipoproteinA-1 is administered in the form of a sterile liquid pharmaceutical formulation. The may be administered alone, or in combination with other substances, such as phospholipids. In an embodiment, the pegylated apolipoproteinA-1 is administered with lipid, such as a phospholipid. In an embodiment the phsopholipid is 1-palmitoyl-2-oleoyl-sn-glycero-3-phosphocholine (also termed, 1-palmitoyl-2-oleoyl-phosphatidylcholine or POPC). The phsopholipids can be one or more of the phospholipid can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives. The administration of the other biologically active substances can be concurrent or sequential with administration of pegylated apolipoproteinA-1. The pegylated apolipoprotein A-1 and lipids can be mixed in an aqueous solution in appropriate ratios and can be complexed by methods known in the art including freeze-drying, detergent solubilization followed by dialysis, microfluidization, sonication, and homogenization. The amount of pegylated apoA-1 to lipid (including phospholipids) can be 100:1, about 10:1, about 5:1, about 3:1, about 2:1, about 1:1, about 1:2, about 1:3, about 1:5, about 1:10 and about 1:100 (wt of protein/wt of lipid). In addition, the recited HDL particles comprising the mono-pegylated apoA-I can comprise one or more of the phospholipids recited herein.

In compositions comprising both mono-pegylated and multi-peglyated apoA-1 the proportion of each type of pegylated apoA-1 can be determined by routine techniques such as size separation, western blots and chromatography.

In an embodiment the pegylated apolipoproteinA-1 is administered as a liquid pharmaceutical formulation comprising less than 6,000 particulates greater than 10 pm in size per 50 mL. In an embodiment the pegylated apolipoproteinA-1 is administered as a liquid pharmaceutical formulation comprising less than 600 particulates greater than 25 μm in size per 50 mL.

In an embodiment the pegylated apolipoproteinA-1 is administered as a liquid pharmaceutical formulation having an osmolality of about 280 to about 320 mOsm. In an embodiment the pegylated apolipoprotein A-1 is administered as a liquid pharmaceutical formulation having an osmolality of about 290 mOsm.

One method of delivery of mono-pegylated ApoA-I (wild type or mutants) is how ApoA-I is applied currently as a therapeutic agent, through intravenous infusion of large quantities of protein to patients as described by Chiesa G. and Sirtori C R, Curr Opin Investig Drugs March 2002; 3(3):420 6, and Sirtori, C R, et al., Atherosclerosis 142 (1999) 29 40, which are hereby incorporated by reference in their entirety. The mono-pegylated apoA-I (wild type or mutants) can be administered singly or in combination with other cardiovascular or triglyceride-lowering drugs, for example statins. They may be conventionally prepared with excipients and stabilizers in sterilized, lyophilized powdered forms for injection, or prepared with stabilizers and peptidase inhibitors of oral and gastrointestinal metabolism for oral administration. They may also be administered by methods including, but not limited to, intravenous, infusion, or intramuscular administration. In one embodiment, the pegylated ApoA-I is administered by intravenous infusion into a peripheral vein of a subject. In embodiments the pegylated ApoA-I (whether alone or in a pharmaceutical composition) is administered intravenously into the fossa of the arm or a central line into the chest. In embodiments, the pharmaceutical formulation is infused into the cephalic or median cubital vessel at the antecubital fossa in the arm of a subject.

As used herein, a “pharmaceutical carrier” is a pharmaceutically acceptable solvent, suspending agent or vehicle, for delivering the instant compounds to the animal or human. The carrier may be liquid, aerosol, gel or solid and is selected with the planned manner of administration in mind. In an embodiment, the pharmaceutical carrier is a sterile pharmaceutically acceptable solvent suitable for intravenous administration.

In an embodiment the sterile liquid pharmaceutical formulation comprises a sucrose-mannitol carrier and a phosphate buffer. In an embodiment the sucrose-mannitol carrier comprises about 6.0% to about 6.4% sucrose and about 0.8% to about 1% mannitol. In an embodiment the sucrose-mannitol carrier comprises about 6.2% sucrose and about 0.9% mannitol. In an embodiment the sterile liquid pharmaceutical formulation has a pH of about 7.0 to about 7.8. In an embodiment the sterile liquid pharmaceutical formulation has a pH is about 7.5. Formulations and methods of administration of apolipoproteinA-1 to subjects are described in U.S. Pat. No. 7,435,717 which is hereby incorporated in its entirety, and which formulations and methods may be used for the pegylated apolipoproteinA-1 described herein.

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In an embodiment, the subject being treated has a HDL-cholesterol level of below 45 mg/dl for a man or below 50 mg/dl for a women. In an embodiment the subject being treated (male or female) has a HDL-cholesterol of <40 mg/dL [<1.04 mmol/L].

The pegylated apolipoproteinA-1 and compositions, and HDL particles comprising such, in the methods of treatment described herein are used in effective amounts. As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Treatment of the diseases recited herein, e.g. of a cardiovascular disease, encompasses inducing inhibition, regression, or stasis of the disorder. In an embodiment, treatment of diseases having atheroma as a symptom thereof comprises reduction in the volume of the atheroma. Non-limiting examples include athermoa and atherosclerosis.

Polyethylene glycol, unless otherwise stated, means, in regard to a pegylated molecule, a polymer having the formula OH—CH₂—(CH₂—O—CH₂)ₙ—CH₂—OH, wherein one of the hydroxy groups is replaced by a covalent bond to the molecule being pegylated and wherein n is the number of oxyethylene groups. The polyethylene glycol can have an average molecular weight of 20,000, wherein the actual molecular weight is no less than 90% of and no greater than 110% of the average molecular weight. In an embodiment, the PEG is a branched PEG. In embodiments the PEG can be straight chain, substituted or unsubstituted.

Source of polyethylene glycol shall mean any art-recognized source of PEG for purposes of pegylating a molecule. Non-limiting examples include methoxy PEG (e.g. in the form of methoxy PEG propionaldehyde, of molecular weight 20,000 available from JenKem Technology USA, Allen, Tex.). Other sources include Polyethylene glycol 200, 300, 400, 600, 1000, 1450, 3350, 4000, 6000, 8000, 20000, 30000 and 40000 (CAS No.: 25322-68-3), and tresyl monomethoxy PEG (TMPEG). Branched and multiarm PEG may also be used. Pegylation techniques are discussed in Roberts et al., Adv Drug Deliv Rev. 2002 Jun. 17;54(4):459-76, hereby incorporated by reference in its entirety. Activated PEGS that target —NH₂, —OH or —SH can be used.

In an embodiment, the PEGylated molecule is made as follows: PEG aldehyde was stored at −20° C. and taken from storage and fully equilibrated to room temperature before use. Human apoA-I was reconstituted in 50 mM sodium acetate, pH 5.5, 10 mM sodium cyanoborahydride at concentration of −4 mg/ml. The amount of PEG aldehyde to be used (molar ratio PEG:apoA-I=10:1) was calculated and dissolved in the buffer used 50 mM sodium acetate, pH 5.5, 10 mM sodium cyanoborahydride. Then, PEG solution was added slowly to protein solution with gentle swirling. The mixture was incubated at 4° C. overnight. The reaction was quenched with addition of glycine to 10 mM and incubated for 4 hours at 4° C. The pegylated human apoA-I samples were partially purified with a desalting column.

As used herein “about” with regard to a stated number encompasses a range of +one percent to −one percent of the stated value. By way of example, 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

Where a range is given in the specification it is understood that the range includes all integers and 0.1 units within that range, and any sub-range thereof. For example, a range of 77 to 90% includes the times 77, 78, 79, 80, and 81% etc.

The treatment with the pegylated apolipoproteinA-1 may be a component of a combination therapy or an adjunct therapy, i.e. the subject or patient in need of the drug is treated or given another drug for the disease in conjunction with one or more of the instant compounds, e.g. statins, niacin, estrogen, ezemtimibe, nicotinic acid. This combination therapy can be sequential therapy where the patient is treated first with one drug and then the other or the two drugs are given simultaneously. These can be administered independently by the same route or by two or more different routes of administration depending on the dosage forms employed.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

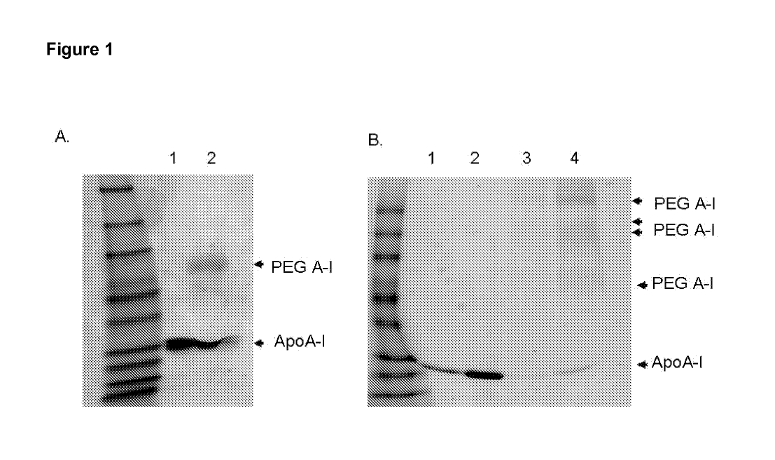

1. Pegylation of human apoA-I. Methoxy PEG Propionaldehyde (M-PEG-ALD), MW 20000 was purchased from JenKem Technology USA (Allen, Tex.). The PEG-containing bottle was removed from storage and fully equilibrated to room temperature. ApoA-I reconstituted in 50 mM sodium acetate, pH 5.5, 10 mM sodium cyanoborahydride was dissolved. PEG Aldehyde was used at a molar ratio of 10:1, PEG:apoA-I. The required amount of PEG was dissolved in an aliquot of 50 mM sodium acetate, pH 5.5, 10 mM sodium cyanoborahydride. The PEG preparation was mixed with the human apoA-I solution with gentle swirling. The final concentration of human apoA-I was about 2 to 6 mg/ml. The mixture was incubated at 4° C. overnight. To minimize the formation of multipegylated species, the reaction was quenched by adding 1 M glycine to a concentration of 10 mM or to a concentration of 50 mM. Under these conditions, apoA-I was preferentially pegylated at the N-terminus. The pegylated human apoA-I was analyzed by SDS-PAGE and Coomassie Brilliant Blue staining and shown in FIG. 1. After −16 hours incubation at 4° C., approximately half of human apoA-I molecules were pegylated (FIG. 1A). The pegylated apoA-I appeared to be largely a homologous species under these conditions. If incubation temperature was changed to room temperature, a large portion of apoA-I was pegylated (FIG. 1B). However, multipegylation of apoA-I appeared to be dominant under these conditions because multiple species of PEG-apoA-I were generated with higher molecular weight (FIG. 1B).

Purified human apoA-I can also be pegylated predominantly at its N-terminus with M-PEG-ALD of MW 5000, 30000 or 40000. The M-PEG-ALD is obtained from a suitable supplier, e.g. JenKem Technology USA (Allen, Tex.). After being fully equilibrated to room temperature from −20° C. storage it is dissolved in an aliquot of 50 mM sodium acetate, pH 5.5, 10 mM sodium cyanoborahydride, and the M-PEG-ALD is then immediately added in a molar ratio of 10:1 to human apoA-I reconstituted in 50 mM sodium acetate, pH 5.5, 10 mM sodium cyanoborahydride with gentle swirl. The final concentration of human apoA-I is about 5 mg/ml. The mixture is incubated at 4° C. for 16 hours. At the end of incubation, the reaction was quenched by addition of an aliquot of 1 M glycine solution to the mixture to make the final concentration 50 mM. The PEG-apoA-I preparation will be subjected to SDS-PAGE and Coomassie Brilliant Blue staining for analysis of the pegylation efficiency. The unmodified control apoA-I will be processed similarly and incubated in the same buffer without M-PEG-ALD. At the end of the incubation, glycine-quenched M-PEG-ALD is added to the native human apoA-I preparation. The preparation is used as the control in SDS-PAGE and Coomassie Brilliant Blue staining analysis as well as in the macrophage cholesterol efflux assay as detailed below.

To show that the pegylated apoA-I is pegylated at its N-terminus but not C-terminus, the control and PEG-apoA-I are subjected to limited proteolysis by proteases. C-terminal and N-terminal specific anti-apoA-I antibodies are used to determine the size of the different apoA-I polypeptide fragments. A size change is seen of the N-terminal fragment but not of the C-terminal fragment after N-terminal preferential pegylation. Further, N-terminal pegylation is seen to block N-terminal protein sequence analysis. PEG-apoA-I isolated from the SDS-PAGE gel is subjected to N-terminal protein sequence analysis to determine if the N-terminus is blocked. The unmodified native apoA-I is used as the positive control. ApoA-I is processed similarly except that quenched PEG is added at the end of the incubation will be used as an additional control to test if the processing condition without pegylation will modify the N-terminus of apoA-I and block its N-terminal protein sequencing. The N-terminal apoA-I protein sequencing will be blocked after N-terminal preferential pegylation. However, N-terminal apoA-I protein sequencing will not be affected for the unmodified human apoA-I.

2. Pegylated human apoA-I is biologically active and promotes cholesterol efflux from macrophage foam cells.

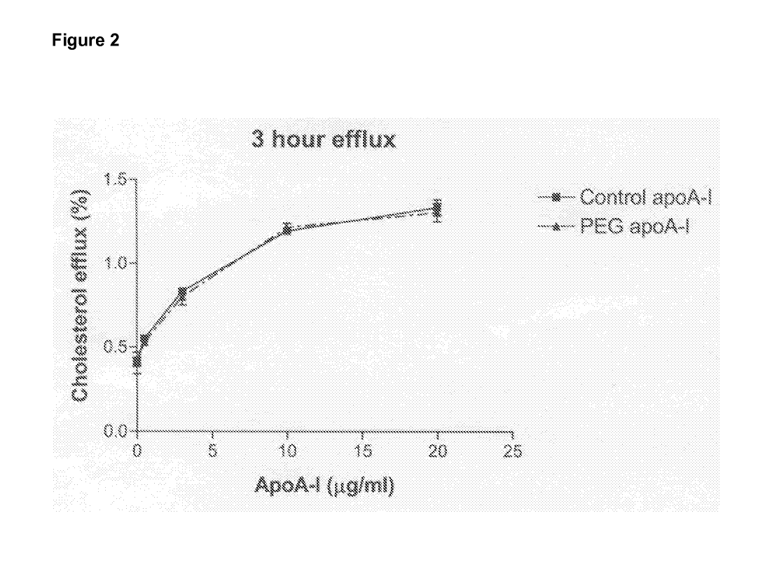

A major mechanism proposed for the anti-atherogenic property of apoA-I and HDL is reverse cholesterol transport, in which HDL transports cholesterol from the peripheral tissue such as macrophage foam cells back to the liver for disposal. In this scenario, cholesterol efflux from macrophage foam cells to apoA-I or HDL constitutes the initial step of reverse cholesterol transport. It has been well documented that apoA-I and HDL act as cholesterol acceptors and promote cholesterol efflux from macrophage foam cells. Therefore, it is important to test the biological activity of apoA-I to promote cholesterol efflux after pegylation of the molecule. It is not clear if this functional activity would be retained in the presence of the pegylation. Mouse peritoneal macrophages were loaded with cholesterol by incubating the cells with a modified LDL preparation (100 μg acetyl-LDL protein/ml) in the presence of radioactive cholesterol tracer ([³H]cholesterol). The cells were washed three times with the last wash after equilibrium in cell culture media plus 0.2% bovine albumin for 30 minutes. Cholesterol efflux was then initiated by addition of unmodified native apoA-I or pegylated apoA-I preparation. In order to rule out the potential effect of free PEG molecules in the efflux assay, the same amount of quenched PEG preparation was added to the group of unmodified apoA-I preparation during the efflux assay. The results are shown in FIG. 2. As shown, pegylation of human apoA-I did not adversely affect the activity of apoA-I to promote cholesterol efflux. Cholesterol efflux from macrophage foam cells to apoA-I or PEG-apoA-I showed a similar dose response (FIG. 2). The PEG-apoA-I preparation used in the efflux assays in FIG. 2 was from the preparations as shown in FIG. 1A. Multipegylation of apoA-I as shown in FIG. 1B, however, caused decrease of cholesterol efflux from macrophage foam cells (not shown).

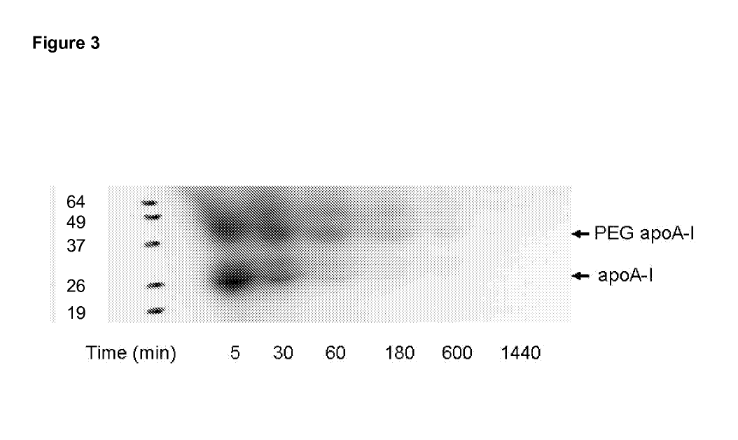

3. Pegylated apoA-I has an increased half life in animal models. In order to evaluate the pharmacokinetics and clearance of PEG-apoA-I in plasma, the PEG-apoA-I preparation, which contained approximately equal amounts of unmodified native apoA-I and PEG-apoA-I (FIG. 1A), was labeled with [¹²⁵I] by iodination. The labeled apolipoprotein preparation was then injected into the blood circulation of the wild type C57BL6/J mice via tail vein. Blood samples were taken from the mice periodically over a 24 hour period. The blood samples were subjected to SDS-PAGE and an autoradiogram was generated and shown in FIG. 3. The results showed that PEG-apoA-I had a reduced plasma clearance relative to that of apoA-I.

For a more protracted analysis, an aliquot of human native apoA-I and PEG-apoA-I mixture will be injected intravenously into C57BL6/J mice. An aliquot of plasma will be obtained periodically over 72 hours and the content of human native or PEG-apoA-I will be determined. Two methods will be used to determine the plasma clearance of human apoA-I. One is using the iodinated human [¹²⁵I]apoA-I as illustrated in FIG. 3. The second method is to use a specific anti-human apoA-I antibody. This antibody will specifically recognize human but not mouse apoA-I. Therefore, it can be used to precisely determine the plasma clearance rate of human apoA-I in mice with Western blot analysis. Alternatively, an ELISA kit specific for human apoA-I can be used to determine the level of plasma human apoA-I in mice.

An important determinant in plasma clearance of human apoA-I is the dose of human apoA-I. The preliminary studies above showed that human PEG-apoA-I could be recovered largely from the lipoprotein fractions 30 minutes after injection into mouse blood circulation. Since previous studies indicate that apoA-I added to the plasma in vitro could readily incorporate into preformed lipoprotein particles, it suggests that a portion of the human PEG-apoA-I rapidly incorporates into existing lipoprotein particles. Therefore, the capacity of existing lipoprotein particles in plasma to accommodate lipid-poor apoA-I could be rate-limiting in determine the half life of human native or PEG-apoA-I as the lipid-poor apoA-I could have different plasma half life as compared with apoA-I incorporated into lipoprotein particles. Different doses of human PEG-apoA-I in plasma half-life determination are determined to optimize the 2.5 dose using unmodified human apoA-I as the control. In normal C57BL6/J mice, the average mouse plasma apoA-I levels are −30 mg/dL (equivalent to −13 mg/kg body weight). Thus, a dose range from 5 to 80 mg/kg body weight can be used. This is compatible to the recombinant HDL dose used in human studies (up to 80 mg/kg body weight).

The plasma samples taken from these studies are alternatively used for analysis of other parameters.

These include lipid (cholesterol, phospholipid and triglycerides) and lipoprotein (VLDL, LDL and HDL) profile analysis, the distribution of human apoA-I in different lipoprotein or plasma fractions, macrophage cholesterol efflux to the total plasma or distinct lipoprotein fractions as well as routine blood or plasma chemical or cell analysis.

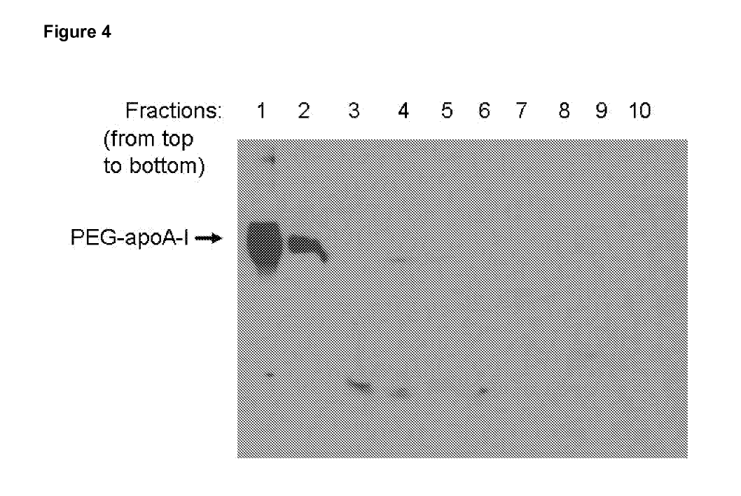

4. PEG-apoA-I readily incorporates into plasma HDL. It is important to understand if the fate of PEG-apoA-I is into HDL particles or other lipoprotein particles, or does it quickly generate lipoprotein particles by interacting with other gene products like ABC transporters? A blood sample was taken from a mouse 30 minutes after injection of PEG-apoA-I into the blood circulation of the mouse and the plasma samples were then subjected to a density gradient centrifugation which has been routinely used as a well-documented method to isolate lipoproteins from the plasma. Following the centrifugation, different fractions from the top to the bottom of the centrifuge tube were loaded to the electrophoresis gel and characterized by Western immunoblot analysis to determine the distribution of PEG-apoA-I in the plasma. As shown in FIG. 4, most of the PEG-apoA-I was recovered in the top fractions after centrifugation, indicating that PEG-apoA-I is either rapidly incorporated into existing lipoprotein particles or generates new lipoprotein particles in vivo. Pegylation of apoA-I is a preferred method to produce a novel form of apoA-I with increased half-life in plasma but which retains its biological activities. Therefore, pegylated apoA-I is an alternative choice to increase the efficacy and decrease the toxicity of human apoAI—or HDL-mediated therapy for cardiovascular or inflammatory diseases, but the extent of the pegylation is critical.
5. Atherosclerosis Prevention and Treatment. LDLR deficient mice are used as the model for these studies. Alternatively, apoE deficient mice can be used. 2 month old female mice (15 mice for each group) are maintained on the high fat/high cholesterol diet for 9 weeks to generate early atherosclerotic lesions. Then, placebo, human apoA-I or human PEG-apoA-I are given intravenously for a total of 15 tri-weekly injections at a high and a low dose. While tail vein injection is an option, recent studies suggest that retro-orbital venous sinus appears to be a better choice. The mice are maintained on the high fat high cholesterol diet for the five week period. During this period and at the end of the experiment, blood samples are collected weekly to monitor the plasma lipid and lipoprotein profiles. Mice are then sacrificed for atherosclerotic lesion analysis. The atherosclerosis lesion is analyzed using a standard protocol established in this lab. Cross sections from the proximal aorta are embedded in paraffin and the lesion area will be quantified by morphometric analysis of hematoxylin and eosin (H&E)-stained sections with Image-Pro Plus v4.1 software (Media Cybernetics, Bethesda, Md.). Immunostaining will be performed with antibodies used to detect macrophages (Mac-3, BD PharMingen, San Diego, Calif.), smooth muscle cells (-actin, Zymed Laboratories, Sal Francisco, Calif.), endothelial cells (M-20, Santa Cruz Biotechnologies, Santa Cruz, Calif.). Collagen is detected by Masson's trichrome stain (Poly Scientific, Bay Shore, N.Y.).

In addition, other parameters are determined: monocytosis, plasma concentration of pro-inflammatory cytokines (IL-1β, IL-6, MCP-1, TNFα). The plasma collected is used for cholesterol efflux assays.

Administration of pegylated ApoA-1 as described herein is found to reduce atherosclerosis development and incidence.

### EXAMPLES

Subjects having an inflammatory vascular disease are intravenously administered a pegylated apolipoproteinA-1 described hereinabove over a period of one month to two months at a dose of 15 mg/kg to 45 mg/kg of apolipoproteinA-1. Treatment progress can be measured by standard ultrasound methods. The pegylated apolipoproteinA-1 is administered in a sterile liquid pharmaceutical formulation comprising a sucrose-mannitol carrier and a phosphate buffer. The sucrose-mannitol carrier comprises about 6.2% sucrose and about 0.9% mannitol. The sterile liquid pharmaceutical formulation has a pH of about 7.5. The liquid pharmaceutical formulation can have an osmolality of about 290 mOsm. In cases where the inflammatory vascular disease is atheroma, a reduction in atheroma volume is observed. In cases where the inflammatory vascular disease is atherosclerosis, a decrease in plaque size is observed.

Subjects having an inflammatory vascular disease are intravenously administered a composition comprising high density lipoprotein (HDL) particles which HDL comprises pegylated apolipoproteinA-1 described hereinabove over a period of one month to two months. Treatment progress can be measured by standard ultrasound methods. The pegylated apolipoproteinA-1 is administered as an HDL particle liquid pharmaceutical formulation comprising less than 6,000 particulates greater than 10 μm in size per 50 mL or less than 600 particulates greater than 25 μm in size per 50 mL. In cases where the inflammatory vascular disease is atherosclerosis, a decrease in plaque size is observed. In cases where the inflammatory vascular disease is atheroma, a reduction in atheroma volume is observed.

Subjects having a dyslipidemia are intravenously administered a composition comprising high density lipoprotein (HDL) particles which HDL comprises pegylated apolipoproteinA-1 described hereinabove over a period of one month to two months. The administration of the pegylated apoA-1 comprising HDL composition is found to ameliorate the dyslipidemia in the subjects.

Intravenous administration of a composition comprising high density lipoprotein (HDL) particles which HDL comprises pegylated apolipoproteinA-1 described hereinabove or of the pegylated apolipoproteinA-1, over a period of one month to two months is found to increase plasma HDL levels in subjects having a HDL-cholesterol level of below 45 mg/dl for a man or below 50 mg/dl for a women. The pegylated apolipoproteinA-1-containing HDL composition is administered as an HDL particle liquid pharmaceutical formulation comprising less than 6,000 particulates greater than 10 μm in size per 50 mL or less than 600 particulates greater than 25 μm in size per 50 mL.

### Discussion

While pharmacologic intervention to treat atherosclerosis traditionally focused on lowering LDL-cholesterol levels as a therapeutic target, a number of intervention trials have also shown that there is an inverse correlation between the plasma concentration of HDLs and the incidence of cardiovascular disease. The epidemiologic evidence in support of a cardioprotective function for HDL-cholesterol suggests that HDLs influence the cellular processes involved in atherogenesis. Therefore, high-density lipoprotein (HDL) therapy has drawn increased attention from researchers, and has shown great potential to become a new approach that is complementary to existing therapies for atherosclerosis. However, one problem concerning the direct infusion of HDL, recombinant apo-AI or apo-AI-phospholipid complexes is achieving and maintaining plasma recombinant HDL at its sufficient therapy concentration over an appropriate period of time without causing adverse effects in humans. In addition, making suitable HDL particle compositions without bile salts has not been attempted. It is not known if pegylation of apolipoproteins can increase their plasma half-life, while still permitting retention of their biological functions. In addition, it is also not known if such pegylated particles can still form HDL particles. Finally, it is not known if pegylation of apolipoproteins will prevent the apolipoprotein from promoting cholesterol efflux from macrophage cells.

Disclosed here is a method using pegylation of human apoA-I with activated methoxypoly(ethylene glycol) (MPEG) to increase the plasma half life of human apoA-I. Importantly, the mono-pegylated form of human apoA-I made still retains its biological activities such as promotion of cholesterol efflux from macrophage foam cells. Surprisingly, the degree of pegylation is critical, with applicants observing that multi-pegylated apoA-1 proved to have qualities the opposite of that desired. The mono-pegylated apoA-1 has an increased half life, cholesterol promoting activity and ability to form HDL particles. The multi-pegylated form decreased cholesterol efflux from macrophages when tested. The pegylated form of human apoA-I is a more efficient form of human apoA-I for apoA-I or HDL therapy for cardiovascular and/or inflammatory diseases. In addition, applicants have formulated a method of producing the HDL particles which does not use bile salts, which salts can cause adverse health effects in humans.

1. An isolated mono-pegylated apolipoproteinA-1.

2. The isolated mono-pegylated apolipoproteinA-1 of claim 1, wherein the apolipoproteinA-1 comprises consecutive amino acids having the sequence set forth in SEQ ID NO:1.

3. The isolated mono-pegylated apolipoproteinA-1 of claim 1, wherein the apolipoproteinA-1 comprises consecutive amino acids encoded by the nucleic acid sequence set forth in SEQ ID NO:2 or SEQ ID NO:3.

4. The isolated mono-pegylated apolipoproteinA-1 of claim 1, wherein the polyethylene glycol has a molecular weight of between 19,000 and 21,000.

5. The isolated mono-pegylated apolipoproteinA-1 of claim 1, wherein the polyethylene glycol has a molecular weight of 20,000.

6. The isolated mono-pegylated apolipoproteinA-1 of claim 1, wherein the apolipoproteinA-1 is N-terminally mono-pegylated.

7. The isolated mono-pegylated apolipoproteinA-1 of claim 1, wherein the apolipoproteinA-1 is A-1 Milano (Arg173Cys)_or the variant (Arg173Pro).

8. A composition comprising a carrier and a total molar amount of mono-pegylated apolipoproteinA-1, wherein the total molar amount of mono-pegylated apolipoproteinA-1 is more than 50% relative to the total molar amount of apolipoproteinA-1, wherein the total molar amount of apolipoproteinA-1 includes mono-pegylated apolipoproteinA-1, multi-pegylated apolipoproteinA-1, and non-pegylated apolipoproteinA-1.

9-14. (canceled)

15. The composition of claim 8, wherein the composition is substantially free of multi-pegylated apolipoproteinA-1.

16. The composition of claim 8, wherein the total molar amount of mono-pegylated apolipoproteinA-1 is more than 75% relative to the total molar amount of apolipoproteinA-1.

17-29. (canceled)

30. A composition of claim 8 comprising a high-density lipoprotein particle which comprises a pegylated apolipoproteinA-1, wherein the composition is essentially free of bile salts.

31-44. (canceled)

45. A process for preparing an isolated mono-pegylated apolioprotein-A-1, the method comprising:
(a) admixing apolipoproteinA-1 with a source of polyethylene gylcol under conditions permitting formation of a covalent bond between the apolipoproteinA-1 and the polyethylene glycol; and
(b) separating mono-pegylated apolioprotein-A1 from the product of step (a) or quenching step (a) before production of multi-pegylated apolipoproteinA-1 occurs, so as to thereby prepare isolated mono-pegylated apolioprotein-A1.

46-80. (canceled)

81. A method of treating an inflammatory vascular disease in a subject comprising administering to the subject an amount of the compound of claim 1 effective to treat the inflammatory vascular disease in the subject.

82-86. (canceled)

87. A method of treating a dyslipidemia in a subject comprising administering to the subject an amount of the compound of claim 1 effective to treat the dyslipidemia in the subject.

88. A method of increasing plasma high-density lipoprotein levels in a subject comprising administering to the subject an amount of the compound of claim 1 effective to increase plasma high-density lipoprotein levels in the subject.

89. A method of promoting cholesterol efflux from macrophage foam cells in a subject comprising administering to the subject an amount of the compound of claim 1 effective to promote cholesterol efflux from macrophage foam cells in the subject.

90. A method of treating an inflammatory vascular disease in a subject comprising administering to the subject an amount of the compound of claim 8 effective to treat the inflammatory vascular disease in the subject.

91. A method of treating a dyslipidemia in a subject comprising administering to the subject an amount of the compound of claim 8 effective to treat the dyslipidemia in the subject.

92. A method of increasing plasma high-density lipoprotein levels in a subject comprising administering to the subject an amount of the compound of claim 8 effective to increase plasma high-density lipoprotein levels in the subject.

93. A method of promoting cholesterol efflux from macrophage foam cells in a subject comprising administering to the subject an amount of the compound of claim 8 effective to promote cholesterol efflux from macrophage foam cells in the subject.


SEQUENCE LISTING (3 sequences)

SEQ ID NO: 1 - protein, 267 aa, homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - DNA, 897 bp, homo sapiens
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 3 - DNA, 926 bp, homo sapiens
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtgcgaaaaaaaaaaaaaaaaaaaaaaaaaaaaa